(19) United States
(12) Patent Application Publication
(10) Pub. No.: US 2018/0050071 A1
(43) Pub. Date: Feb. 22, 2018

(54) ANTI-AGE COMPOSITION COMPRISING A COMBINATION OF ANTIOXIDANT AGENTS IN ASSOCIATION WITH BIFIDOBACTERIA AND CELL WALLS ISOLATED FROM PROBIOTICS

(71) Applicant: BIOIMMUNIZER SAGL, Lugano (CH)

(72) Inventor: Bernard BIZZINI, Lugano (CH)

(21) Appl. No.: 15/556,803

(22) PCT Filed: Mar. 9, 2016
(86) PCT No.: PCT/IB2016/000251

§ 371 Date: Sep. 8, 2017

(30) Foreign Application Priority Data
Mar. 9, 2015 (IT) 102015902336785

(51) Int. Cl. A61K 35/745; A61K 33/30; A61K 33/04; A61K 9/00; A61K 31/221; A61K 36/87; A61K 36/45; A61K 36/81; A61K 36/9066; A61K 31/07; A61K 31/375; A61K 31/355; A61K 31/198; A61K 8/27; A61K 8/23; A61K 8/99; A61K 8/44; A61K 8/9789; A61K 8/9794; A61K 8/67; A61K 8/46; A61Q 19/08; A23L 33/105; A23L 33/135; A23L 33/00; A23L 33/15; A23L 33/16

(52) U.S. Cl. CPC A61K 35/745; A23V 2002/00; A61K 33/04; A61K 9/0014; A61K 9/0053; A61K 31/221; A61K 36/87; A61K 36/45; A61K 36/81; A61K 36/9066; A61K 31/07; A61K 31/375; A61K 31/355; A61K 31/198; A61K 8/27; A61K 8/23; A61K 8/99; A61K 8/44; A61K 8/9789; A61K 8/9794; A61K 8/671; A61K 8/676; A61K 8/678; A61K 8/46; A61Q 19/08; A23L 33/105; A23L 33/135; A23L 33/30; A23L 33/15; A23L 33/16; A61K 2800/5922; A61K 33/30

(57) ABSTRACT

An anti-age composition is described. The anti-age composition comprises a combination of antioxidant agents, such as flavonoids, anthocyanins, tannins, curcumin and derivatives thereof, as well as vitamins A, C and E, in association with Bifidobacteria and cell walls isolated from probiotics which takes into account all the different factors involved in the aging process in order to prevent or slow down the course thereof.

### SUMMARY OF THE INVENTION

The object of the present invention is an anti-age composition comprising a combination of antioxidant agents in association with Bifidobacteria and cell walls isolated from probiotics, which takes into account all the different factors involved in the aging process in order to prevent or slow down the course thereof. Cell homeostasis is under the control of redox systems. An unbalance of such a control leads to the release of ROS (Reactive Oxygen Species) and RNS (Reactive Nitrogen Species), which causes severe damages to cells, thereby triggering the aging process. Under physiological conditions antioxidant substances are naturally produced, such as, for example, the superoxide dismutase, being able to counteract the detrimental action of ROS and RNS to the cells.

In order to fight the detrimental effects of ROS and RNS, which are key players in the aging process, substances, such as flavonoids, anthocyanins, tannins, curcumin and derivatives thereof, as well as vitamins A, C and E were found to be able to inactivate free radicals and, subsequently, the detrimental effects thereof.

Moreover, it has been fully demonstrated that homeostasis of intestinal flora is fundamental for individual's well-being. In particular, the persistence of a high amount of Bifidobacteria, as major agents of the beneficial action on the intestinal flora is well proved. On the other hand, it is well known that a strong relationship between metabolism and immune system exists.

### BACKGROUND

The continuous and countless attacks from both the external environment (among others, microbes, bacteria) and those induced inside the body (over-produced mediators, catabolic derivatives, etc.) to which the body is subjected cause, over time, a weakness, or even an exhaustion, of the immune defence which usually allows to resist these attacks. Particular attention has been paid to the detection of factors involved in the aging process. Among these factors, free radicals derived from oxygen by forming Reactive Oxygen Species (ROS) or from nitrogen, by forming Reactive Nitrogen Species (RNS) have been shown to be key players. Usually, cell homeostasis is under the control of redox systems, which ensure a balance between oxidants and antioxidants (Pacher P, Beckman J S, Liaudet L et al. Nitric oxide and peroxynitrite in health and disease, Physiol. Rev., 2007, 87, 315-424). When said balance is altered, the over-produced ROS and RNS dramatically impair the functioning of cells until their killing or allowing the occurrence of diseases. The “free radicals” theory of aging has been firstly suggested by Harman (Harman D., Aging: a theory based on free radical and radiation chemistry, J. Gerontology, 1966, 11, 298-300). This theory has been expanded by Hakim (Hakim S, Lapointe J, Wen Y. Taking, A “good look” at free radicals in the aging process, Trends in Cell Biology, 2011, 21, 569-576) and by Halliwell (Halliwell B, Free radicals and antioxidants updating a personal view, Nutrition Reviews, 2012, 70, 257-265).

Furthermore, this theory includes also diseases resulting from the action of free radicals (Harman D., Origin and evolution of the free radical theory of aging: a brief personal history, 1954-2009. Biogerontology, 2009, 10, 773-781).

Miguel et al. showed that mitochondria have a key role in the free radical theory (Miguel J, Economos A C, Fleming G et al. Mitochondrial role in cell aging. Exp. Gerontol., 1980, 15, 575-591). ROS can damage the mitochondrial DNA and proteins, and the resulting injuries further enhance the production of ROS derivatives. In this way a positive “feedback loop” of oxidative stress occurs, which firstly leads to a damage of cells, organs and finally of the overall body (Afanas'ev., Signaling and damaging function of free radicals in aging-free radical theory, hormesis and tor., Aging and Disease, 2010, 1, 75-88).

The antioxidant administration has been suggested to be able to complexing the oxidant molecules thereby reducing the damages caused to the cells, which are responsible for aging process and disease occurrence (Biesalski H, Free, Radical theory of aging., Current Opinion in Clinical Nutrition and Metabolic Care, 2002, 5, 5-10). In fact, antioxidants are substances with reducing power, able to limiting oxidative damages to the biological structures by complexing free radicals (Halliwell B, Free radicals and antioxidants: updating a personal view, Nutrition Reviews, 2012, 70, 257-265).

Substances with anti-oxidant activity are naturally produced in order to maintain the redox system balance within cells. Natural antioxidants are mainly superoxide dismutases (SODs), catalases, glutathione and glutatione peroxidases and reductases.

However, in order to be successful, the fight against aging should not be limited to passivation of free radicals. It has been found that intestinal flora is directly involved in body's well-being. Intestinal flora effects have to be ascribed to the ability of intestinal bacteria to stimulating the digestion, food catabolism, peristalsis, to providing energy, amino acids and vitamins, to inactivating or degrading toxic products and, rather, to building a barrier against pathogenic bacteria (Coates M E, Fuller R, Harrison G F. Intestinal synthesis of vitamins of the B complex in chicks., Brit. J. Nutr., 1968, 22, 493), (Coates M E, The influence of the gut microflora on the nutrition of its hosts., Bibli. Nutr. Dietan, 1975, 22, 101-108), (Brown J P., Role of gut bacterial flora in nutrition and health., Crit. Rev. Food Sci. Nutr., 1977, 8, 229-336).

The intestinal flora contains 100 billions of microbes, consequently it has been considered as a microbial organ inside an organ (intestine) of the host, consisting of different kinds of microorganisms with specific metabolic functions, being able to communicate with each other and the host (Backhed F, Ley R I, Sonnenburg J L et al. Host-bacterial mutualism in the human intestine., Science, 2005, 307, 1915-1920). The intestinal microflora is formed during the birth when the fetus meets for the first time the microorganisms colonizing the mother's vaginal mucosa (Tissier H. Répartition des microbes dans I'intestin du nourrisson. Ann Inst. Pasteur, 1905, 19, 109-114) (Ducluzeau R, Moreau C, Duval I et al. L'établissement de la flore microbienne dans le tube digestif du nouveau'né., Rev. Int. Pediatr, 1982, 120, 13-18).

The stable indigenous flora being formed consists of about 150 bacterial species, of which only 10 species are in a large amount, with a prevalence of Bifidobacteria.

Under healthy conditions, the microflora stability depends on its intrinsic resistance to external attacks, which prevents the establishment of novel bacterial strains. Colonization of intestinal flora by Bifidobacteria is not long-lasting and accounts for an external supply of such bacteria for thereby maintaining their sustained beneficial effect throughout the life.

The evidence that a modification of intestinal flora composition is related to obesity and type 2 diabetes proves the relevance of a stable flora (Everard A, Belzer C, Geurts L et al. Cross-Talk between Akkermansia muciniphila and intestinal epithelium controls diet-induced obesity, PNAS, 2013; 110, 9066-9071).

It has also been reported that a high-fat diet leads to a reduction of Bifidobacteria population in the flora, whereas the increase of this population is related to an enhanced tolerance to glucose, insulin secretion in response to glucose and a lowering of the body weight and a less production of inflammatory mediators (Cani P D, Neyrinck A M, Knauf C et al., Selective increases of Bifidobacteria in gut microflora improve high-fat-diet-induced diabetes in mice through a mechanism associated with endotoxaemia, Diabetologia, 2007, 50, 2374-2383).

Immune system and metabolism are known to be strongly related.

The link existing between them contributed to the understanding of the pathogenesis of metabolic diseases, for the treatment of which immunomodulation could be used (Osborn or, Olefsky I M. The cellular and signaling networks linking the immune system and metabolism in disease, Nature Medicine, 2012, 18, 363-374) and (Maslowski K M, Mackay C R., Diet, gut, microbiota and immune responses, Nat. Immun., 2011, 12, 5-9) and (Kau A S, Ahern P P, Griffin M W et al. Human nutrition, the gut microbiome and the immune system, Nature, 2011, 474, 327-336).

Sekine et al. reported that preparations of cell walls from Bifidobacterium infantis exert a strong antitumor activity (Sekine K, Toida T, Salto L et al. A new morphologically characterized cell wall preparation (whole peptidoglycan) from Bifidobacterium infantis with a higher efficacy on the regression of an established tumor in mice, Cancer Res., 1985, 45, 1300-1307).

By applying the method described for preparing cell walls from Corynebacterium granulosum (Bizzini B, Maro B, Lallouette P.Isolement et caractérisation d'una fraction, dite P40, á partir de Corynebacterium granulosum, Med. et Mal. Infect., 1978, 408-414) to a preparation of cell walls from different species of Bifidobacteria and Lactobacilli, cell wall preparations from said different bacteria were obtained, which are characterized by immunostimulant activity, in particular they activate macrophages and restore a weakened immune system due to the administration of cyclophosphamide. On the other hand, the topical application of a gel containing any one of these cell wall preparations to volunteers affected by psoriasis and/or acne resulted in a significant amelioration of lesions.

### OBJECT OF THE INVENTION

It is the object of the present invention a composition for oral or topical use comprising:
one or more natural extracts selected from the group consisting of grape seed extract; grape marc extract; bilberry extract; Goji berry extract; curcuma extract in an amount from 2.5 to 10% by weight relative to the weight of the final formulation;
one or more substances with anti-oxidant activity selected from the group consisting of vitamin A, vitamin C, vitamin E and acetylcysteine in an amount from 0.1 to 1.0% by weight relative to the weight of the final formulation; and/or
zinc and selenium in an amount from 0.005 to 0.02% by weight of the final formulation;
probiotics selected from live bacteria of Bifidobacterium bifidum, in an amount of 10×8-10×10 cfu/g; and/or
cell walls isolated either from Bifidobacterium bifidum, or from Bifidobacterium infantis/longum, or from Lactobacillus aerophilus or from Lactobacillus plantarum in an amount from 0.0025 to 0.025% by weight relative to the final formulation.

Antioxidants are aimed to inactivating over-produced free radicals and preventing them from damaging cells and the function thereof. Given their beneficial effects on health, a high level of Bifidobacteria and the persistence thereof should be maintained in the intestinal flora, since the objective of cell walls isolated from Bifidobacteria with immunostimulant activity is to restoring the immunocompetence, which is often, if not always, weakened in the elderly.

Therefore, it is a major aspect of the present invention the composition according to the invention, due to the fact that it is able to fight at different levels the processes involved in aging.

The Bifidobacterium contained in the compositions being object of the present invention is live, in an amount sufficient for performing the administration at a rate of 10×8 cfu/day. Compositions can be suitably formulated in an amount of at least 10×8 cfu/g, in particular at doses of 10×9 cfu/g, more particularly from 10×9 to 10×10 cfu/g carrier.

In a preferred embodiment of the present invention the compositions for topical application according to the invention do not comprise live bacteria of Bifidobacterium bifidum, but cell walls isolated from any one of the probiotics at a dose from 0.0025 to 0.025% by weight relative to the total weight and preferably at a dose of 50 μg/g of a typical formulation, such as for example a gel.

The preferred antioxidant substances for preparing the composition being object of the present invention are selected from the group consisting of natural extracts: grape seed extract, which supplies flavonoids and proanthocyanidins; grape marc extract, which supplies flavones and anthocyans (tannins); bilberry extract due to its effect on blood microcirculation; Goji berry extract, both from Lycium Barbarum and Lycium chinense due to its very high content of antioxidants and immunostimulant polysaccharides; curcuma extract containing curcuminoid substances well known for their antioxidant effect.

In addition to natural extracts, the compositions, object of the present invention, comprise one or more antioxidant substances selected from the group consisting of vitamins A, C and E due to their known antioxidant effects; and acetylcysteine for its reducing power.

Such antioxidant substances are commercially available or can be prepared according to the methods described in the Experimental Part.

In a preferred embodiment of the present invention the composition, object of the present invention, comprises: natural extracts 10%: bilberry extract 2.5%, Goji extract 5%, curcuma extract 2.5%; vitamins 1% and acetylcysteine 0.1%.

The composition, object of the present invention, is useful for counteracting the aging process in order to prevent or slow down the course thereof.

In particular it is useful for maintaining the proper amount of Bifidobacterium in the intestinal flora, which, as shown above, has major properties as immunomodulator, for preventing diabetes 2, as anti-obesity or anti-tumor composition. Furthermore, the topical administration of the composition, object of the present invention, can be useful in the treatment of dermatological disorders such as psoriasis, acne, atopic dermatitis, atopic eczema as well as dry skin.

The composition, object of the present invention, can be formulated as oral or topical compositions.

By effective amount is meant, according to the present invention, the amount sufficient for obtaining the intended effect.

As described in the present invention, the compositions herein disclosed can comprise compositions in a pharmaceutically acceptable form. The compositions can further comprise, if desired, one or more additional active agents.

The compositions being object of the present invention can be formulated as pharmaceutical, nutraceutical, probiotic, or cosmetic compositions and administered to a mammal, such as a human patient in a variety of forms suitable for the selected administration route, namely, the oral or topical one.

The compositions of the invention can be administered along with other supplements and therapeutic compounds and used in other kinds of treatment.

The present compositions can be systemically administered, e.g. orally, combined with a pharmaceutically acceptable vehicle such as an inert diluent or an edible assimilable vehicle. They can be contained into soft or hard gel capsules, can be compressed into tablets, or can be directly added to the foodstuff in the patient's diet.

For the oral therapeutic administration, the active composition can be directly administered in the form of packets or combined with one or more excipients and directly used as packets or in the form of ingestible tablets, buccal tablets, lozenges, capsules, elixirs, suspensions, syrups, cachets and the like.

Tablets, lozenges, pills, capsules and the like can also contain the followings: binding agents such as tragacanth, acacia, corn starch or gelatin, excipients such as dicalcium phosphate; a disintegrating agent such as corn starch, potato starch, alginic acid and the like; a lubricant such as magnesium stearate; and a sweetener such as sucrose, fructose, lactose or aspartame or a flavoring agent such as peppermint, gaultheria oil, or cherry flavor can be added.

When the unit dosage form is a capsule, it can contain, in addition to the above-cited materials, a liquid vehicle, such as a vegetable oil or polyethylene glycol. Several further materials can be present as coatings or for modifying in a different way the physical form of the solid unit dosage form. For example, tablets, pills, or capsules can be coated with gelatin, wax, shellac or sugar and the like.

A syrup or elixir can contain the active compound, sucrose or fructose as sweetener, methyl and propyl parabens as preservatives, a coloring agent and a flavor such as cherry or orange flavor.

Any material being used in the preparation of any unit dosage form should be pharmaceutically acceptable and substantially non-toxic at the employed amounts. Furthermore, the active compound can be incorporated into sustained-release preparations and devices.

For a topical administration, the present compositions can be applied as formulations, along with a dermatologically acceptable carrier, which can be a solid or a liquid.

Useful solid carriers comprise finely divided solids such as talc, clay, microcrystalline cellulose, silica, alumina and the like. Useful liquid carriers comprise water, alcohol, or glycols or mixtures water-alcohol/glycols, in which the present compounds can be dissolved or dispersed at effective levels, optionally by the aid of non-toxic surfactants. Co-adjuvants such as fragrances can be added, in order to optimizing the properties for an intended use.

Thickening agents such as synthetic polymers, fatty acids, salts and esters of fatty acids, fatty alcohols, modified celluloses or modified mineral materials can also be used with liquid carriers to form spreadable pastes, gels, ointments, soaps and the like, to be directly applied on the recipient's skin.

The useful dosages of compounds of formula I can be determined by comparing their activity in vitro, and in vivo in animal models.

Usually, the concentration of the compositions of the present invention in a liquid composition, such as a lotion, would be of about 0.1-25% by weight, preferably about 0.5-10% by weight. The concentration in a semi-solid or solid composition such as a gel or a powder would be of about 0.1-5% by weight.

The amount of the composition, object of the present invention, required for the use in a treatment will vary depending on the specific combination, as well as based on the administration route, the kind of condition to be treated and the age and status of the patient and will be basically at the discretion of the physician or health care staff assisting the patient.

However, a suitable dose would be generally comprised in the range between about 1.5-25 mg/kg e.g. about 5-25 mg/kg of body weight per day, for example from 3 to about 25 mg per kilogram of body weight of the recipient per day, preferably in the range comprised from 5 to 25 g/kg/day, more preferably in the range from 5 to 25 mg/kg/day.

The compound is advantageously administered in a unit dosage form; for example containing from 5 to 1000 mg, advantageously from 10 to 750 mg, more advantageously from 50 to 500 mg of active ingredient per unit dosage form.

The intended dose can be advantageously presented in a single dose or as multi-doses administered within proper ranges of time, for example, two, three, four or more sub-doses per day.

The composition for topical application does not contain Bifidobacterium bifidum, but cell walls isolated from any one of the probiotics, in addition to antioxidants.

In the context of the present invention, the term “unsaturated fatty acid” relates to a fatty acid comprising at least a double bond.

This is particularly suitable for long-chain fatty acids, i.e., which can have more than 14 carbon atoms.

Unsaturated fatty acids can be in acidic form or salt form, such as calcium salt thereof, or in the form of derivatives, in particular esters.

Fatty acids can be either monounsaturated, such as petroselenic acid (C12), palmitoleic acid (C16) and oleic acid (C18), or polyunsaturated, namely, having at least two double bonds.

The selection of fatty acids is intended to be calculated by taking into account the aim of their composition that means, for topical application or oral administration.

Polyunsaturated fatty acids include ω-3 and ω-6 fatty acids, characterized by the position of the nearest unsaturated terminal methyl group, and mixtures thereof.

Particularly suitable for the compositions, object of the present invention, are the unsaturated fatty acids having from 18 to 22 carbon atoms, in particular polyunsaturated fatty acids, among which ω-3 and ω-6 fatty acids.

Among the polyunsaturated fatty acids of ω-6 series, in particular linoleic acid with 18 carbon atoms and two unsaturated bonds (18:2, ω-6), linoleic acid with 18 carbon atoms and three unsaturated bonds (18:3, ω-6), di-homo-gammalinolenic acid with 20 carbon atoms and 3 unsaturated bonds (20:3, ω-6), arachidonic acid, 5, 8, 11, 14 (eicosatetraenoic 20:4, ω-6) and docosatetraenoic acid (22:4, ω-6) can be mentioned.

Polyunsaturated fatty acids of ω-3 series can be especially selected from alpha-linolenic acid (18:3, ω-3), stearidonic acid (18:4, ω-3), 5,8,11,14,17-eicosapentaenoic acid (EPA 20:5, ω-3) and docosahexaenoic acid 4,7,10,13,16,19 or DHA (22:6 ω-3), docosapentaenoic acid (22:5, ω-3), n-butyl-5,11,14-eicosatrienoic acid.

Particularly suitable for the compositions, object of the present invention, are linolenic acid, stearidonic acid, eicosapentaenoic acid, docosahexaenoic acid, or mixtures or extracts thereof.

The content of unsaturated or polyunsaturated fatty acids and fatty acid esters, in the compositions of the invention can vary from 0.0001% to 90% by weight, especially 0.01-50% by weight, and in particular from 0.1 to 10% by weight relative to the total weight of the composition.

Sources of γ-linolenic acid can be selected from vegetable oils, such as evening primrose oil, borage, blackcurrant seeds, echium and hemp, and extracts from the microalga Spirulina (Spirulina maxima and Spirulina platensis).

Vegetable oils of nut, hazelnut, almond (Juglans regia), coriander and soya (Glycine max), Argan, Cannabis, rape (Brassica naptus), chia, lin, fish oils, for example, are polyunsaturated fatty acids of ω-3 series.

Polyunsaturated fatty acids ω-3 can be also found in zooplankton, shellfish/mollusks and fish.

Fish oils are the main industrial source of EPA and DHA. The microalgal biomass can also be a source of extraction for unsaturated fatty acid ω-3 material.

Thus, the unsaturated fatty acid can be used in the composition in the form of at least an oil selected from evening primrose, borate, blackcurrant seeds, nut, soya, fish, sunflower, wheat germ, hemp, fenugreek, anca, echium, argan, baobab, rice bran, sesame, almond, hazelnut, chia, lin, rose, olive, avocado, safflower, coriander oils and/or microalgal extract (ex. Spirulina), or zooplankton extracts.

The compositions, according to the invention, can comprise such oils and/or extracts and/or biomass in a content from 5 to 80% by weight, in particular from 10 to 70% by weight relative to the total weight of the composition, in particular for oral administration.

The compositions, according to the invention, can comprise such oils and/or extracts and/or biomass in concentrations adjusted such that they are administered in an amount from 0.1 to 10 g/day, in particular from 0.2 g to 5 g/day.

The topical compositions or combinations according to the invention can further contain other ingredients, for example: vitamins B3, B5, B6, B8, C, E, PP or, niacin, carotenoids, polyphenols, and minerals such as zinc, calcium, magnesium.

At least a prebiotic or a prebiotic mixture can be also comprised.

More particularly, these prebiotics can be selected, for example, from oligosaccharides produced from glucose, gelactose, xylose, maltose, sucrose, lactose, starch, xylan, hemicellulose, inulin, acacia gum, or mixtures thereof.

More particularly, the oligosaccharide comprises at least a fructo-oligosaccharide.

More particularly, this prebiotic can comprise a mixture of fructo-oligosaccharides and inulin.

As regards in particular the compositions for topical administration, they can be aqueous solutions, aqueous or oily alcoholic solutions, dispersions of solution type or dispersions such as lotion or serum, liquid or semi-liquid emulsions such as milk, suspensions or emulsions like a cream, aqueous or anhydrous gel, microemulsions, microcapsules, or vesicular dispersions of ionic and/or non-ionic surfactant.

Such compositions are prepared according to standard methods.

These compositions can be used in particular for producing creams for cleansing, protecting, treating or curing the face, hands, feet, large skin folds or the body (for example daily creams, night creams, make-up remover creams, foundations, sun protection creams), beauty products such as liquid foundations, cleansing milk, protective or supporting body milk, after sun milks, lotions, gels or foams for skin care, such as cleansing or disinfectant lotions, sun creams, self-tanning lotions, bath compositions, deodorant compositions containing a bactericidal agent, after-shave gels or lotions, depilatory creams, or insect-repellent compositions.

The compositions of the invention can also be used for producing solid preparations such as soaps.

They can also be used for hair care in the form of solutions, creams, gels, emulsions or mousse, or in the form of aerosol compositions further containing a propellant under pressure.

When the composition of the invention is an emulsion, the proportion of fatty phase can vary from 5 to 80% by weight, and preferably from 5 to 50% by weight relative to the total weight of the composition.

Oils, emulsifiers and co-emulsifiers being used in the composition in the form of emulsion are selected from those conventionally employed in cosmetic and/or dermatological products.

The emulsifier and co-emulsifier can be in the composition in an amount comprised between 0.3 and 30% by weight, and preferably from 0.5 to 20% by weight relative to the total weight of the composition.

When the composition of the present invention is a solution or an oily gel, the fatty phase can accounts for more than 90% of the total weight of the composition.

According to known methods, the cosmetic and/or dermatological composition according to the invention can further contain cosmetic, pharmaceutical and/or dermatological adjuvants, such as hydrophilic or lipophilic agents, hydrophilic or lipophilic gelling active agents, preservatives, antioxidants, solvents, fragrances, bulking agents, screening agents, bactericides, odor absorbers and coloring agents.

The amounts of these various co-adjuvants are those conventionally used in the field, for example 0.01-20% of the total weight of the composition.

Such co-adjuvants, depending on the kind thereof, can be introduced in the fatty phase and/or in the aqueous phase.

As regards fats being used in the invention, in addition to unsaturated fatty acids, mineral oils such as hydrogenated polyisobutene and liquid petrolatum, vegetable oils such as a liquid fraction of shea butter, sunflower and apricot kernel oil, animal oils such as perhydrosqualene, synthetic oils, such as PurCellin oil, isopropyl myristate and octyl palmitate, and fluorine oils such as perfluoropolyether can be used.

Fatty alcohols can also be used, fatty acids such as stearic acid and such waxes comprise paraffin, carnauba and beeswax.

Furthermore, silicone compounds, such as silicone oils and, for example cyclomethicone and dimethicone, waxes, resins and silicone gums can be used.

Emulsifiers used in the present invention include for example glycerol stearate, polysorbate 60, mixture of cetyl alcohol/oxyethylene cetostearyl alcohol with 33 moles of ethylene oxide sold under the name Sinnowax AO by Henkel, PEG-6/PEG-32/glycol stearate sold under the name Tefose 63 by Gattefosse Company, PPG-3 myristyl ether, silicone emulsifiers such as cetyl dimethicone copolyol and sorbitan mono or tristearate, PEG-40 stearate, oxyethylene sorbitan (200E).

As solvents used in the present invention lower alcohols, particularly ethanol and isopropanol, and propylene glycol are comprised.

As hydrophilic gelling agents carboxylic polymers such as carbomer, acrylic copolymers such as acrylate/polyacrylamide copolymers comprising the mixture polyacrylamide, C13-C14 isoparaffins and Laureth-7 sold under the name SEPIGEL 305 by SEPPIC Company, polysaccharides such as cellulose derivatives like hydroxyalkyl cellulose, and in particular hydroxypropyl cellulose and hydroxyethyl cellulose, natural gums such as guar, carob and xanthan gum, and clays are included.

As lipophilic gelling agents modified clays such as bentonites, metal salts of fatty acids such as aluminum stearates and hydrophobic silica, or ethyl cellulose and polyethylene are comprised.

As hydrophilic active agents, proteins or protein hydrolysates, amino acids, C2 to C10 polyalcohols such as glycerin, sorbitol, butylene glycol and polyethylene glycol, urea, allantoin, sugars and sugar derivatives, water-soluble vitamins, starch, bacterial or vegetable extracts such as Aloe Vera can be used.

As lipophilic active agents retinol (vitamin A) and derivatives thereof, tocopherol (vitamin E) and derivatives thereof, ceramides, essential and unsaponifiable oils (tocotrienols, Sesamin, gamma-oryzanol, phytosterols, squalene, waxes, terpenes) can be used.

The activities according to the invention can also be combined with active agents mainly intended for preventing and/or treating skin disorders.

The method for cosmetic treatment of the present invention can be performed in particular by applying the cosmetic and/or dermatological compositions or combinations as defined above, according to the standard technique for the use of said compositions.

For example: the application of creams, gels, serums, lotions, cleansing milk or after sun compositions on dry skin or hair, the application of a hair lotion for wetting the hair, shampoo, or the application of a dentifrice to the gums.

The cosmetic method according to the invention comprises the use, for example a daily use, of the combination according to the invention, which can be for example formulated as gels, lotions, emulsions. The method of the invention can comprise a single administration.

In another embodiment, the administration of the compositions object of the present invention is repeated for example from 2 to 3 times per day for one or more days in general, and for a prolonged period of at least 4 weeks, or even from 4 to 15 weeks, optionally with one or more periods of discontinuation.

The invention is now described with reference to the following Examples and Embodiments. In the absence of an additional description, it is believed that an ordinary skilled in the field can, by using the foregoing description and the following illustrative examples, prepare and use the present invention and follow the described methods.

Thus, the following practical examples, are provided as illustrative only and specifically underline the preferred embodiments of the present invention, and are not intended to be construed in any way as limitative of the description. Therefore, the examples should be understood so that to comprise any variation, which would be obvious as a result of the teaching provided in the present invention which is within the scope of the appended claims.

### EXPERIMENTAL PART

### Example 1

### 1. Culturing of Bifidobacteria

Either B. bifidum (Tissier) {DSMZ no. 20456}, or B. infantis/longum (DSMZ no. 20088) can be used in the composition object of the present invention.

The selected species of Bifidobacterium is cultured on Blaurock medium (Blaurock K G Zuchtung von B. Bifidus bakterien aus der vaginal Flora. Deutsche Medizin Wochenschrift, 1940, 113) supplemented with 0.12% ascorbic acid under strict anaerobic conditions at 38° C.

Bacteria are collected at the beginning of the stationary phase by centrifugation and washed twice by centrifugation after resuspension in a neutralized solution of 1% ascorbic acid. The bacterial precipitate resuspended in a minimum volume of a neutralized solution of 0.1% ascorbic acid is freeze-dried. The Lactobacteria culturing is performed under the same conditions on Scardovi medium.

### 2. Obtaining Cell Walls From Bifidobacteria or Lactobacteria

Cell walls of Bifidobacteria are prepared according to the method by B. Bizzini et al. (Med. Mal. Inf. 1978, 8, 408).

Bacteria are killed by heating for 30 minutes at 60° C. and washed once by centrifugation after resuspension in water. The bacterial mass is dried in a stove at 50° C. before to be delipidated by extraction in a Soxhlet for a 8 hour cycle with a mixture of ethanol:ether 1:1; trichloroethanol; a mixture of methanol:trichloroethanol 2:1. After drying, bacteria are suspended in water and broken by means of a Waring Blender. The unbroken bacteria are removed by centrifugation at 1000×g for 5 minutes. Cell walls are precipitated from the supernatant containing the broken bacteria by adding a saturated ammonium sulfate solution up to 40% of saturation.

The precipitate is allowed to decanting overnight at 4° C. and the cell walls are collected by centrifugation at 10.000×g for 15 minutes. The deposit is resuspended in water and dialyzed against water until the removal of any trace of ammonium sulfate. Then, it is freeze-dried.

### Example 2

### Antioxidants Extraction

The extraction of antioxidants is performed by cold maceration in a mixture of water 70, glycerol 20, ethanol 10, and, separately, of grape, grape marc and bilberry seeds and goji berries for 48-96 hours. The extracts are then distilled to dryness under vacuum in a rotavapor.

The curcuminoid extract is prepared by extraction in a Soxhlet.

The curcuma rhizomes are washed with water and air-stream dried at 50° C. for 6 hours; they are powdered by means of a mill. The powder is extracted with methanol and the extract is distilled to dryness under vacuum in a rotavapor.

### Example 3

### Preparation of the Composition

The composition for the administration as such contained in a packet consists of:

Grape seed extract: 2.5-10%

Grape marc extract, which supplies flavones and anthocyans (tannins); 2.5-10%

Bilberry extract for its effect on blood microcirculation; 1-5%

Goji berry extract; 1-10%

Curcuminoid extract; 1-10%

Vitamins A, C and E; 0.1-1%

Acetylcysteine; 0.1-1%

Zinc and selenium salts; 0.0025-0.025%

Bifidobacteria; 10×8-10×10 cfu/g

Cell walls from Bifidobacteria or Lactobacilli; 0.025-0.2%

1. A composition, formulated for oral or topical administration, the composition comprising:
one or more natural extracts selected from the group consisting of grape seed extract; grape marc extract; bilberry extract; Goji berry extract; curcuma extract; in an amount from 2.5 to 10% by weight relative to the total weight of the composition;
one or more substances with anti-oxidant activity selected from the group consisting of vitamin A, vitamin C, vitamin E and acetylcysteine in an amount from 0.1 to 1.0% by weight relative to the total weight of the composition;
zinc and selenium in an amount from 0.005 to 0.02% by weight relative to the total weight;
probiotics selected from live bacteria of Bifidobacterium bifidum, in an amount of 10×8-10×10 cfu/g; and/or
cell walls isolated from a bacteria selected from the group consisting of Bifidobacterium bifidum, Bifidobacterium infantis/longum, Lactobacillus aerophilus and Lactobacillus plantarum in an amount from 0.0025 to 0.025% by weight relative to the total weight of the composition.

2. The composition according to claim 1, wherein, for a topical application, the composition is devoid of live bacteria of Bifidobacterium bifidum.

3. An oral pharmaceutical formulation comprising the composition according to claim 1 and common excipients.

4. A topical pharmaceutical formulation comprising the composition according to claim 1 and common excipients.

5. A nutraceutical formulation comprising the composition according to claim 1, with or without common excipients.

6. A cosmetic formulation comprising the composition according to claim 1 and common excipients.

7. A method of treating a subject, comprising:
administering to the subject an effective amount of the formulation according to claim 3 and or 4 as drugs.

8. A method comprising:
administering to a subject the formulation according to claim 3 or 4 in an effective amount for maintaining a proper amount of Bifidobacterium in intestinal flora; for preventing diabetes 2, as anti-obesity or anti-tumor formulations; and/or for treatment of a dermatological disorder.

9. The method of claim 8, wherein the dermatological disorder is selected from the group consisting of psoriasis, acne, atopic dermatitis, and atopic eczema dry skin.

10. The composition according to claim 1 comprises the one or more natural extracts in about 10%, vitamins in about 1% and acetylcysteine in about 0.1% by weight of the total weight of the composition.

11. The composition according to claim 1 further comprising prebiotics selected from oligosaccharides produced from glucose, galactose, xylose, maltose, sucrose, lactose, starch, xylan, hemicellulose, inulin, acacia gum, or mixtures thereof.
